(19) United States
(12) Patent Application Publication
(10) Pub. No.: US 2024/0279704 A1
(43) Pub. Date: Aug. 22, 2024

(54) USING FUCOSIDASE TO CONTROL AFUCOSYLATION LEVEL OF GLYCOSYLATED PROTEINS

(71) Applicant: AMGEN INC., Thousand Oaks, CA (US)

(72) Inventors: Namratha SUBHASH, Cambridge, MA (US); Yu Cong CHEN, Seattle, WA (US); Kyle Shamus MCELEARNEY, Newbury Park, CA (US); Glen BOLTON, Boston, MA (US)

(21) Appl. No.: 18/567,747

(22) PCT Filed: Jun. 6, 2022
(86) PCT No.: PCT/US2022/032389

§ 371 Date: Dec. 6, 2023

Related U.S. Application Data
(60) Provisional application No. 63/197,908, filed on Jun. 7, 2021.

(51) Int. Cl. C12P 21/00; C07K 1/22; C07K 1/34; C07K 16/24; C12N 9/24

(52) U.S. Cl. CPC C12P 21/005; C07K 1/22; C07K 1/34; C07K 16/244; C12N 9/2402; C07K 2317/14; C07K 2317/41; C07K 2317/72; C12Y 302/01051

(57) ABSTRACT

Provided herein are methods of obtaining a recombinant glycosylated protein having increased levels of afucosylated glycoforms. In exemplary embodiments, the methods comprise incubating purified recombinant glycosylated protein with a human broad specificity fucosidase and separating the recombinant glycosylated protein from the fucosidase.

### FIELD OF THE INVENTION

The present disclosure relates to the field of glycosylated proteins. In particular, the disclosure relates to methods of obtaining glycosylated proteins, such as antibodies, with increased afucosylation by subjecting purified proteins to a fucosidase and separating the glycosylated protein from the fucosidase.

### BACKGROUND

Recombinant monoclonal antibody (mAb) based therapeutics are established class of biologics that have been introduced for treatment of illnesses like cancer, inflammation and other autoimmune disorders. See, e.g., Sha et al., 2016, Trends Biotechnol 34:835-846. During post translational modification, mAbs undergo glycosylation which is one of the most common and important, yet complex, modification. The complexity of this step stems from the chemical heterogeneity involved with the covalent attachment of sugar (glycan) moieties to protein most commonly at Asn (N-linked) or Ser/Thr (O-linked) residues. N-Linked glycan composition attached to the Fc region is a critical quality attribute for mAbs. Glycosylation plays a role in multiple cellular functions, including, for example, protein folding, quality control, molecular trafficking and sorting, and cell surface receptor interaction. The level of glycosylation affects the therapeutic efficacy of recombinant protein drugs, as it influences the bioactivity, pharmacokinetics, immunogenicity, solubility, and in vivo clearance of a therapeutic glycoprotein. Glycosylation of monoclonal antibodies (mAbs) also affects safety, thus understanding the impact and matching glycosylation profiles is crucial in biosimilar drug development. Glycans are known to impact the antibody-dependent cell-mediated cytotoxicity (ADCC) activities as well as complement-dependent cytotoxicity (CDC) which are key effector functions for mAbs. See, e.g., Liu et al., 2015, J Pharm Sci 104:1866-1884.

One area of glycosylation interest is the modulation of afucosylated species in the final drug substance to optimize the therapeutic function of IgG molecules. Fc glycoform profiles, in particular, are important product quality attributes for recombinant antibodies, as they directly impact the clinical efficacy and pharmacokinetics of the antibodies. The Fc region binds to various cell receptors, such as Fc receptors, and other immune molecules such as complement proteins. This binding mediates processes such as opsonization, cell lysis and degranulation of mast cells, basophils and eosinophils. See Woof et al., 2004, Nat Rev Immunol 4:89-99. The absence of the monosaccharide fucose attached to the core glycan structure was found to contribute in enhancing the ADCC function and binding affinity of the therapeutic. See Zhang et al., 2016, MAbs 8:205-215.

During upstream production, the glycan profile on mAbs can be varied by the varying of the cell line, process conditions, media and feed formulations and genetic engineering in earlier development stages. See, e.g., Ehret et al., 2019, Biotechnology and Bioengineering 116:816-830. The effect of these variables in modulating the glycosylation levels is complicated and difficult to implement considering the complications involved during the cell's uptake, growth and harvest.

A number of different approaches to reduce fucosylation have been described. U.S. Pat. Nos. 10,407,673; 10,077,434; 9,540,673; 8,642,292; 8,409,838; 7,919,313; and 7,214,775; U.S. Patent Application Publication Nos. US2020/0199236; US2019/0185898 US2019/0112358; US2018/0251572; and US2018/0171028; and International Patent Application Publication Nos. WO2020/042015; WO2020042022; and WO2019/246383 describe host cells with modification of enzymes in the fucosylation pathway or knock outs of genes encoding enzymes in the fucosylation pathway such as a fucosyltransferase (FUT8). U.S. Pat. Nos. 10,676,772; and 10,167,492; U.S. Patent Application Publication No. US2020/0131518; and International Patent Application Publication Nos. WO2020/033827; WO2020/094694; WO2019/224333; WO2019/191150; and WO2018/114929 describe controlling fucosylation by modifying cell culture conditions. U.S. Pat. No. 9,504,702; and International Patent Application Publication No. WO2019/196697 describe the use of fucose or mannose analogs to inhibit fucosylation. U.S. Pat. No. 9,096,877 describes engineering Fc-region amino acid sequences to create mutations which attenuate post-translational fucosylation. U.S. Pat. No. 10,087,236 describes stepwise modification of the Fc glycosylation pattern of a human, chimeric or humanized antibody where one step uses an alpha-fucosidase and other steps use a glycosyltranserase, such as endo-β-N-acetyl-glucosaminidase, or alpha-2,6-sialyltransferase.

While there are many ways to influence the levels of particular glycoforms of an antibody, there still is a need in the biopharmaceutical industry for simple and efficient methods to manipulate and control the levels of total afucosylated glycoform during recombinant production of therapeutic antibodies.

### SUMMARY

The present disclosure provides a method for obtaining a recombinant glycosylated protein having increased levels of afucosylated glycoforms, the method comprising a) incubating a purified recombinant glycosylated protein with a human broad specificity fucosidase in a buffer suitable for fucosidase activity for a time and under conditions suitable to increase afucosylation of the recombinant glycosylated protein; and b) separating the recombinant glycosylated protein having increased levels of afucosylated glycoforms from the fucosidase; wherein the recombinant glycosylated protein is not reacted with a glycosyltransferase or sialyltransferase.

In certain embodiments, the human fucosidase is α-(1-2,3,4,6)-L-fucosidase. In certain embodiments, the fucosidase is present at a level between 1000 U/mmol to 100,000 U/mmol recombinant glycosylated protein. In certain embodiments, the fucosidase is present at a level between 5,000 U/mmol to 25,000 U/mmol recombinant glycosylated protein.

In certain embodiments, the incubating is for between 1 hour to 24 hours

In certain embodiments, the buffer has a pH from about 4.0 to about 5.0. In certain embodiments, the buffer is sodium acetate, phosphate buffered saline (PBS) or 2-(N-morpholino)ethanesulfonic acid (MES)

In certain embodiments, the temperature is selected from a temperature between 30° C. and 40° C. In certain embodiments, the temperature is selected from a temperature between 35° C. and 38° C.

In certain embodiments, the purified recombinant glycosylated protein is in an amount greater than or equal to 10 g/L.

In certain embodiments, the fucosidase is immobilized on a solid phase, such as a protein A chromatography resin.

In certain embodiments, the purified recombinant glycosylated protein has been purified by one or more chromatography steps.

In certain embodiments, the levels of one or more of A1G0, A2G0, A2G1a, A2G1b, A2G2, and A1G1M5 of the recombinant glycosylated protein are increased. In certain embodiments, the levels of high mannose (HM) glycoforms of the recombinant glycosylated protein are decreased. In certain embodiments, the levels of one or more of Man5, Man6, Man7, Man8, and/or Man9 of the recombinant glycosylated protein are decreased. In certain embodiments, the percent galactosylation is reduced. In certain embodiments, the percent sialylation is reduced.

In certain embodiments, the recombinant glycosylated protein is separated from the fucosidase using one or more purification steps. In certain embodiments, the one or more purification steps are selected from diafiltration, ultrafiltration, and sterile filtration.

In certain embodiments, the recombinant glycosylated protein is an antibody, a peptibody, or a Fc-fusion protein. In certain embodiments, the recombinant glycosylated protein is an antibody that binds to CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79α, CD79β, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, CD182, erythropoietin, IL-2, IL-3, IL-4, IL-5, IL-6, IL-7, IL-9, IL-11, IL-13, G-CSF, IL-15, GM-CSF, OSM, IFNgamma, IFNα, IFNβ, TNFα, TNFβ, LTβ, CD40 ligand, Fas ligand, CD27 ligand, CD30 ligand, 4-BBL, TGFβ, IL-1α, IL-1β, IL-1 RA, IL-10, IL-12, MIF, IL-16, IL-17, IL-18, glucagon receptor, IL-17 receptor A, Sclerostin, IGF-1 receptor, myostatin, epidermal growth factor receptor, SARS coronavirus, OPGL, Angiopoietin-2, NGF, TGF-β type II receptor, connective tissue growth factor, properdin, CTLA-4, interferon-gamma, MAdCAM, amyloid, insulin-like growth factor I, interleukin-1β, c-Met, M-CSF, MUC18, interleukin-4 receptor, fibroblast growth factor-like polypeptides, α-4β-7, Activin Receptor-like Kinase-1, Activin A, angiopoietin-1, angiopoietin-2, C-FMS, galanin, insulin like growth factor, LDCAM, DKK1, osteoprotegerin, OV064, PSMA, PAR2, HEPCIDIN, B7L-1, c-Kit, ULBP, TSLP, SIGIRR, HER-3, ataxin-1-like polypeptide, TNF-α converting enzyme, IL1-R1, TGF—B type II receptor, TNF receptor-like molecules, connective tissue growth factor, TRAIL receptor-2, erythropoietin receptor, B7RP1, properdin, RANKL, carbonic anhydrase IX (CA IX) tumor antigen, parathyroid hormone, ACPL, monocyte chemo-attractant protein-1, SCF, 4-1BB, PDGFD, Flt-3 ligand, metalloproteinase inhibitor, LERK-5, LERK-6, brain-derived neurotrophic factor, epithelium-derived T-cell factor, neurotrophic factor NNT-1, proprotein convertase subtilisin kexin type 9 (PCSK9), IL-18 RECEPTOR, or C-FMS. In certain embodiments, the recombinant protein is one of Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), Ibritumomab tiuxetan (product marketed with the brand name Zevalin®), Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), Pertuzumab (product marketed with the brand name Perjeta®), adalimumab, infliximab, etanercept, golimumab, certolizumab pegol; canakinumab; ustekinumab, briakinumab; daclizumab, belimumab; epratuzumab; daclizumab; iratumumab, gemtuzumab, alemtuzumab; ipilimumab; cetuximab; trastuzumab, pertuzumab; siltuximab; bevacizumab; and tocilizumab.

### BRIEF DESCRIPTION OF THE DRAWINGS

FIGS. 1A-B show (A) the level of afucosylation (%) or (B) the percent change in afucosylation, plotted on the y-axis. The x-axis shows each fucosidase: Hfuc, BKF and FucO for 24 hr in comparison to the control sample without any fucosidase at 0 hr and 24 hr.

FIGS. 2A-C show the effect of fucosidases on (A) the level of high mannose (%); (B) percent galactosylation; or (C) percent sialylation, plotted on the y-axis. The x-axis shows each fucosidase: Hfuc, BKF and FucO for 24 hr in comparison to the control sample without any fucosidase at 0 hr and 24 hr.

### DETAILED DESCRIPTION

The present invention is based, in part, that the discovery that human α-(1-2,3,4,6)-L-fucosidase, without any other fucosylation pathway enzymes (such as glycosyltranferase or sialyltransferase), can increase afucosylation in purified IgG1 antibodies. Glycoenzymes, which add or cleave glycan residues on substrates, can be used to directly manipulate glycosylation of mAbs during isolation and purification stages of the downstream process. The application of these enzymes during the downstream processing stage evades the complexity and variability of the intracellular glycosylation pathway involved during the growth of mammalian cells. Use of glycoenzymes can enable closer innovator matching and assist in early selection of clones with higher titer or identical quality attributes. One such glycoenzyme is the fucosidase enzyme which belongs to the glycosyl hydrolase family 29 and 95 (GH29 and GH95) that enables cleaving of fucose residues from substrates.

Successful removal of the core fucose on N-linked glycoproteins with a broad-specificity fucosidase from Omnitrophica bacterium has been demonstrated. See Vainauskas et al., 2018, Nature, 8:9504, α1-2,4,6 Fucosidase O is capable of cleaving α1-2, α1-4 and α1-6 linked core fucose residues from N-glycans, thereby increasing the percentage of afucosylated species.

Surprisingly, a study using three fucosidases, from Homo sapiens, Bovine Kidney, or Omnitophica bacterium, tested on fucosylated mAbs, only the α-(1-2,3,4,6)-L-fucosidase from Homo sapiens showed promising results. While this demonstrates that core fucose residues can be cleaved off from an intact glycosylated mAb, the presence of the other core and branched glycan residues potentially causing steric hindrance interferes with the activity of fucosidases from other species.

Thus, in the downstream antibody production process, the human enzyme fucosidase can act as a powerful modulator of fucosylation by cleaving core fucose in the final drug substance, thereby increasing the percentage of afucosylated species. This can be done without the aid of other enzymes involved in glycoslation such as glycosyltransferases and sialyltransferases. One option for modulating afucosylated species is to use commercially available fucosidase in the downstream process.

A fucosidase is an enzyme that breaks down fucose residues from a glycan. In certain embodiments of the disclosure, the fucosidase is a fucosidase from a mammal, preferably a primate, more preferably a human. In certain embodiments of the disclosure, the fucosidase is a broad spectrum fucosidase, for example, an α-(1-2,3,4,6)-L-Fucosidase. In certain embodiments, the fucosidase is from the GH29 family of fucosidases.

Representative fucosidases include enzyme entry EC 3.2.1.51, encoded by the FUCA1 gene, encoding protein sequences including GenBank Accession Nos. NP_000138.2, XP_005245878.1, XP_011539469, and XP_016856394.1.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The invention provided herein relates to methods of increasing afucosylation levels of a protein during recombinant production by glycosylation-competent cells. Without being bound to a particular theory, it is believed that the methods disclosed herein provide a means for compositions comprising higher levels of afucosylation of a given recombinant protein.

As used herein, the term “afucosylated glycoform” or “afuco glycoform” or “afucosylated glycan” or “Afuco” or “AF” or “final afucosylated” refers to glycoforms which lack a core fucose, e.g., an α1,6 linked fucose on the GlcNAc residue involved in the amide bond with the Asn of the N-glycosylation site. Afucosylated glycoforms include, but are not limited to, A1G0, A2G0, A2G1a, A2G1b, A2G2, and A1G1M5. Additional afucosylated glycans include, e.g., A1G1a, G0[H3N4], G0[H4N4], G0[H5N4], FO-N[H3N3]. See, e.g., Reusch and Tejada, 2015, Glycobiology 25(12):1325-1334.

As used herein, the term “high mannose” or “HM” or “final HM” encompasses glycoforms comprising 5, 6, 7, 8, or 9 mannose residues.

In exemplary aspects, the level of afucosylated glycans and amount of HM glycoforms is determined via HILIC. After enzyme cleavage of the N-glycans, HILIC is performed to obtain a chromatogram with several peaks, each peak of which represents a mean distribution (amount) of a different glycoform.

For these purposes, % Peak Area=Peak Area/Total Peak Area×100%, and % Total Peak Area=Sample Total Area/Total Area of the Standard×100%. The calculations used for purposes of determining the % glycoforms may be carried out as follows:

$$\% Afusylated glycoforms = \% A 1 G 0 + \% A 2 G 0 + \% A 2 G 1 a + \% A 2 G 1 b + \% A 2 G 2 + \% A 1 G 1 M 5.$$

$$\% High mannose glycoforms = \% Man 5 ( if detectable ) + \% Man 6 ( if detectable ) + \% Man 7 ( if detectable ) + \% Man 8 ( if detectable ) + \% Man 9 ( if detectable )$$

“Fucosylation” refers to the degree and distribution of fucose residues on polysaccharides and oligosaccharides, for example, N-glycans, 0-glycans and glycolipids. Therapeutic glycoproteins, e.g., antibodies or Fc fusion proteins, with non-fucosylated, or “afucosylated” N-glycans exhibit dramatically enhanced antibody-dependent cellular cytotoxicity (ADCC) due to the enhancement of FcγRIIIa binding capacity without any detectable change in complement-dependent cytotoxicity (CDC) or antigen binding capability. In certain situations, e.g., cancer treatment, non-fucosylated or “afucosylated” antibodies are desirable because they can achieve therapeutic efficacy at low doses, while inducing high cellular cytotoxicity against tumor cells, and triggering high effector function in NK cells via enhanced interaction with FcγRIIIa. In other situations, e.g., treatment of inflammatory or autoimmune diseases, enhanced ADCC and FcγRIIIa binding is not desirable, and accordingly therapeutic glycoproteins with higher levels of fucose residues in their N-glycans can be preferable. As used herein, the term “% afucose” refers to the percentage of non-fucosylated N-glycans present on a recombinant glycoprotein of interest. A higher % afucose denotes a higher number of non-fucosylated N-glycans, and a lower % afucose denotes a higher number of fucosylated N-glycans.

“Sialylation” refers to the type and distribution of sialic acid residues on polysaccharides and oligosaccharides, for example, N-glycans, O-glycans and glycolipids. Sialic acids are most often found at the terminal position of glycans. Sialylation can significantly influence the safety and efficacy profiles of these proteins. In particular, the in vivo half-life of some biopharmaceuticals correlates with the degree of oligosaccharide sialylation. Furthermore, the sialylation pattern can be a very useful measure of product consistency during manufacturing. The two main types of sialyl residues found in biopharmaceuticals produced in mammalian expression systems are N-acetyl-neuraminic acid (NANA) and N-glycolylneuraminic acid (NGNA). These usually occur as terminal structures attached to galactose (Gal) residues at the non-reducing terminii of both N- and O-linked glycans.

“Galactosylation” refers to the type and distribution of galactose residues on polysaccharides and oligosaccharides. Galactose refers to a group of monosaccharides which include open chain and cyclic forms. An important disaccharide form of galactose is galactose-alpha-1,3-galactose (α-gal).

The invention provides a method of obtaining a recombinant glycosylated protein with increased levels of afucosylated glycoforms. In exemplary aspects, the recombinant glycosylated protein is produced by glycosylation-competent cells in a cell culture known to those skilled in the art. The recombinant glycosylated protein is preferably purified from the cell culture harvest using one or more steps including centrifugation and column purification prior to reaction with a fucosidase.

Units of fucosidase: One Unit of α-L-fucosidase activity is defined as the amount of enzyme required to release one μmole of p-nitrophenol (pNP) per minute from p-nitrophenyl-α-L-fucopyranoside (1 mM) in sodium acetate buffer (100 mM) at pH 4.0.

In exemplary embodiments, the method comprises incubating the purified glycoprotein with a fucosidase at a pH from about 2.5 to 5.0. For example, the pH is 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In exemplary aspects, the pH is greater than about 3.0 and less than about 5.0. In exemplary aspects, the pH is greater than about 4.0 and less than about 5.0. In exemplary aspects, the pH is 4.0, 4.1, 4.2, 4.3, 4.4, 4.5, 4.6, 4.7, 4.8, 4.9 or 5.0. In exemplary aspects, the pH is 4.0 or 5.0.

In exemplary embodiments, the method comprises incubating the glycoprotein with a fucosidase for a specified reaction time. In exemplary aspects, the reaction time is about 3 hours to 6 days (e.g., about 3, 4, 6, 9, 12, 15, 18, 21, or 24 hours or about 2, 3, 4, 5, or about 6 days). In exemplary aspects, the reaction period is about 1 day.

In exemplary embodiments, the method further comprises incubating the reaction at a temperature between 25° C. and 60° C. In exemplary embodiments, the temperature is between about 30° C. to about 50° C., between about 35° C. to about 50° C., between about 35° C. to about 40° C. In certain embodiments, the temperature is 36° C., 37° C., or 38° C., or a range of 36° C.±1° C., 37° C.±1° C., or 38° C.±1° C.

In exemplary embodiments, the reaction occurs in 100 mM sodium acetate puffer (pH 4.0 to pH 5.0). Other suitable buffers include, but are not limited to, Phosphate Buffered Saline (PBS) and MES.

In certain embodiments, the volume of the reaction is from 1 ml to 500 ml. In certain embodiments, the volume of the reaction is from 1 ml to 250 ml. In certain embodiments, the volume of the reaction is from 1 ml to 200 ml. In certain embodiments, the volume of the reaction is from 1 ml to 100 ml. In certain embodiments, the volume of the reaction is about 1 ml, 5 ml, 10 ml, 25 ml, 50 ml, 100 ml, 200 ml, 250 ml or 500 ml.

In certain embodiments, the fucosidase or recombinant glycosylated protein can be immobilized on a column to provide precise control of pH, residence time, temperature, and other factors. In one embodiment, the methods of the invention encompass affinity chromatography using a solid support to isolate the mAb that is then enzymatically modified in a single step as it is bound to the support. Affinity chromatography columns are known to those of skill in the art incorporating a column or other type of solid support. The method may employ a variety of conventional solid phase extraction devices, such as small chromatography columns, spin columns, or pipette tips. The column is typically packed with a solid or stationary phase or medium (which may collectively be referred to as the “solid phase”), as is done for conventional affinity chromatography.

The solid phase comprises a molecule chosen for its specific biological interaction with the target mAb and is referred to herein as the “affinity ligand.” Any ligand that has affinity towards antibodies can be used for these methods. Affinity ligands for use in the method of the invention include Protein A, a surface protein from the cell wall of Staphylococcus bacterium and Protein G, a cell surface protein from Streptococcus bacterium. Such ligands that have affinity for immunoglobulins include but not limited to: Protein A (native, recombinant), Protein G (native, recombinant and synthetic), Protein A-G fusion protein, Protein L. These are available from various commercial sources including but not limited to Sigma-Aldrich and Repligen.

The affinity ligands have to be immobilized on to solid media that is retained in the device during purification and modification process. The solid media include but not limited to agarose, sepharose, polyacrylic, polystyrine and other synthetic polymers which provide negligible nonspecific adsorption of non-target proteins and enzymes of modification. The affinity ligand is covalently linked to the solid support by, for example any of a variety of chemistries, such as N-hydroxysuccinimide (NHS) esters, epoxide, aldehyde, or cyanogen bromide, to a solid phase. Such conjugation chemistries are well-known in the art, as exemplified in Hermanson, G. T., Bioconjugate Techniques, Academic Press (Amsterdam, the Netherlands, Ed. 2008) and Wong, S., Chemistry of Protein Conjugation and Cross-Linking, CRC Press (Boca Raton, Fla., 1991).

The immobilized forms of protein A, Protein G, Protein A-G, Protein L and antibody fragments to agarose or sepharose or other matrices are commercially available from various sources, including but not limited to Sigma-Aldrich, ThermoFisher Scientific and GE Healthcare, for capturing and purifying antibodies. The devices for the modification can be easily designed using commercially available empty columns for affinity chromatography depending on the scale of the product needed. The buffer exchanges in these columns can be done by either gravity flow or centrifugation or by pump. Such empty columns are commercially available from various sources including but not limited to ThermoFisher Scientific and Bio-Rad Laboratories

In embodiments of the method of the invention, the columns utilized are microspin columns with immobilized protein A which has strong affinity towards immunoglobulin proteins. The optimization of buffer and incubation conditions is important to obtain desired result to perform the modifications. The column with immobilized affinity ligand is washed with a wash buffer prior to the loading with the selected mAb solution containing the heterogeneous population of mAbs with various Fc region glycan structures. After a period of incubation, the column is again washed prior to the application of an optimized reaction buffer that contains the reactant mixture (one or more of enzymes, cofactors and nucleotide sugars). After a further period of incubation at temperatures of about 30° C. to about 40° C., in aspects about 36-37° C., the column is once again washed with the wash buffer and then elution buffer is applied that releases the modified mAb with desired glycosylation. An optional neutralization buffer as is understood by one of skill in the art can then be used to obtain a final pH of about 7.2.

The wash buffer is designed to maintain high affinity between antibodies and affinity ligands during washings. PBS with pH of about 7.2 can be used as wash buffer, however it is understood by one of skill in the art that the pH may vary to some degree. The wash and reaction buffers are designed to maintain high affinity between antibodies and affinity ligands and, at the same time, retain the activity of reaction enzymes. The wash and reaction buffers are used at temperatures of about 30° C. to about 40° C., and any temperature therein between. Temperatures of about 37° C. are often used. The optimum pH range for high affinity of antibodies to protein A, protein G and protein A/G is about 6.0 to about 8.0. Within this range of pH, the buffers overlap with optimum pH ranges of the affinity ligands can be used in the method of the invention. These include but are not limited to TRIS buffer, BIS-TRIS buffer, MES buffer, BES buffer, MOPS buffer and HEPES buffer.

Washing conditions for the affinity column minimizes non-specific binding and thus negatively affect enzyme reaction and thus mAb modification. Wash conditions are such that they will not break the bind between the affinity ligand and the target mAb.

In exemplary embodiments, the methods of the invention relate to increasing the levels of afucosylation of a protein produced by cells in a cell culture. In exemplary aspects, the levels of one or more of A1G0, A2G0, A2G1a, A2G1b, A2G2, and A1G1M5 of the recombinant glycosylated protein are increased, relative to the control cell culture. In exemplary aspects, the levels of one or more of A1G1a, G0[H3N4], G0[H4N4], G0[H5N4], and FO-N[H3N3] of the recombinant glycosylated protein are increased, relative to the control cell culture.

In certain embodiments, the methods disclosed herein produce a glycoprotein with increased afucosylation, while decreasing one or more of high mannose percentage, percent galactosylation, and percent sialylation.

As used herein, the term “increase” and words stemming therefrom may not be a 100% or complete increase. Rather, there are varying degrees of an increase of which one of ordinary skill in the art recognizes as having a potential benefit. Particularly, in the case of glycosylated proteins, even small changes can have a significant effect on activity. In this respect, the methods described herein may increase the afucosylated glycoform levels to any degree or level, relative a control cell culture. In exemplary embodiments, the increase provided by the methods of the invention is at least or about a 1% increase (e.g., at least or about a 2% increase, at least or about a 3% increase, at least or about a 4% increase, or at least or about a 5% increase), relative a control cell culture. In exemplary embodiments, the level of afucosylated glycoforms of the protein increases by at least about 1.5-fold, relative a control cell culture. In exemplary embodiments, the level of afucosylated glycoforms of the protein increases by at least about 2-fold, relative a control cell culture. In exemplary embodiments, the level of afucosylated glycoforms of the protein increases by at least about 3-fold, relative a control cell culture. In exemplary embodiments, the level of afucosylated glycoforms of the protein increases by at least about 4-fold or 5-fold, relative a control cell culture.

Regarding the methods described herein, the increase affected by such methods are relative to a “control” or a “control cell culture”. The terms are used interchangeably herein. In exemplary aspects, the control is the level of afucosylated glycoforms of the protein when the steps of the inventive method are not carried out. In exemplary aspects, the control is the level of afucosylated glycoforms of the protein when a known method of recombinant production is carried out. As used herein, the term “control cell culture” means a cell culture maintained in the same manner as the cell culture on which the steps of the inventive method are carried out (e.g., cell culture of the inventive method) prior to treatment with fucosidase.

Various methods are known in the art for assessing glycoforms present in a glycoprotein containing composition or for determining a glycoform profile of a particular sample comprising glycoproteins. Suitable methods include, positive ion MALDI-TOF analysis, negative ion MALDI-TOF analysis, weak anion exchange (WAX) chromatography, normal phase chromatography (NP-HPLC), exoglycosidase digestion, Bio-Gel P-4 chromatography, anion-exchange chromatography and one-dimensional NMR spectroscopy, and combinations thereof. See, e.g., Mattu et al., 1998, J Biol Chem 273: 2260-2272; Field et al., 1994, Biochem J 299(Pt 1): 261-275; Yoo et al., 2010, MAbs 2(3): 320-334; Wuhrer et al., 2005, Journal of Chromatography B, 825:124-133; Ruhaak, 2010, Anal Bioanal Chem, 397:3457-3481 and Geoffrey et al., 1996, Analytical Biochemistry 240:210-226. Also, the examples set forth herein describe a suitable method for assessing glycoforms present in a glycoprotein containing composition.

### Purification

In certain embodiments, the recombinant glycosylated protein is purified prior to incubation with a fucosidase. For the purification of antibodies or antibody fragments, which have been produced e.g. by cell cultivation methods, generally a combination of different chromatography steps can be employed. Normally an (protein A) affinity chromatography is followed by one or two additional separation steps. In one embodiment the additional chromatography steps are a cation and an anion exchange chromatography step or vice versa. The final purification step is a so called “polishing step” for the removal of trace impurities and contaminants like aggregated immunoglobulins, residual HCP (host cell protein), DNA (host cell nucleic acid), viruses, or endotoxins. In the methods of the invention, incubation with fucosidase can occur after any of these chromatography/separation steps. The separation of the reacted recombinant glycosylated protein having increased levels of afucosylated glycoforms from the fucosidase can occur by the steps following use of the fucosidase. For example, if the recombinant glycosylated protein is reacted with fucosidase after protein A chromatography and/or ion exchange chromatography, the reacted recombinant glycosylated protein can be then subjected to the polishing step. If the recombinant glycosylated protein is subjected to a polishing step prior to reaction with fucosidase, then the fucosidase can be separated by diafiltration/ultrafiltration or by any other known separation means.

In certain embodiments, the recombinant glycosylated protein is obtained from production by glycosylation-competent cells. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel et al., 2013, Front Immunol 4: 217. In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolate reductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells. See, e.g., Gaillet et al., 2007, Biotechnol Prog 23:200-209; and Khan, 2013, Adv Pharm Bull 3(2): 257-263.

In exemplary aspects, the glycosylation-competent cells are eukaryotic cells. In exemplary aspects, the eukaryotic cells are mammalian cells. In some aspects, the mammalian cells are non-human mammalian cells. In exemplary aspects, the non-human mammalian cells are selected from the group consisting of CHO cells, CHO derivatives (e.g., CHO-K1, CHO pro-3), mouse myeloma cells (e.g., NS0, GS-NS0, Sp2/0), cells engineered to be deficient in dihydrofolate reductase (DHFR) activity (e.g., DUKX-X11, DG44), green African monkey kidney cells (e.g., COS cells, VERO cells), mouse brain tumor cells CAD, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells. Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidase II, calnexin/calreticulin, glycosyltransferases, mannosidases, GlcNAc transferases, galactosyltransferases, and sialyltransferases.

The disclosure also provides methods of preparing a composition comprising increased afucosylated glycoforms of a glycosylated protein produced by cells in a cell culture. In exemplary embodiments, the method comprises (i) maintaining a cell culture at an initial pH for an initial cell culture period, (ii) expanding the cell culture, (iii) collecting the supernatant of the cell culture comprising the protein produced by the cells, (iv) incubating the purified glycosylated protein with a human broad specificity fucosidase in a buffer suitable for fucosidase activity for a time and under conditions suitable to increase afucosylation of the glycosylated protein; and (v) separating the recombinant glycosylated protein having increased levels of afucosylated glycoforms from the fucosidase. In certain embodiments, the recombinant glycosylated is never reacted with a glycosyltransferase or a sialyltransferase.

The method may comprise one or more steps for purifying the protein from a cell culture or the supernatant thereof and preferably recovering the purified protein. In exemplary aspects, the method comprises one or more chromatography steps, e.g., affinity chromatography (e.g., protein A affinity chromatography), ion exchange chromatography, hydrophobic interaction chromatography. In exemplary aspects, the method comprises purifying the protein using a Protein A affinity chromatography resin.

In exemplary embodiments, the method further comprises steps for formulating the purified protein, etc., thereby obtaining a formulation comprising the purified protein. Such steps are described in Formulation and Process Development Strategies for Manufacturing, eds. Jameel and Hershenson, John Wiley & Sons, Inc. (Hoboken, NJ), 2010.

The method can also comprise one or more upstream steps prior to the cell culture steps. In exemplary embodiments, the method comprises steps for generating host cells that express the protein. For example, the methods comprise, in some instances, introducing into host cells a vector comprising a nucleic acid comprising a nucleotide sequence encoding the protein.

Cell culture may be maintained according to any set of conditions suitable for recombinant protein production. For example, the cell culture may be maintained at a particular cell density, culture volume, dissolved oxygen level, pressure, osmolality, and the like. In exemplary aspects, the cell culture prior to inoculation is shaken (e.g., at 70 rpm) at 5% CO2 under standard humidified conditions in a CO2 incubator. In exemplary aspects, the cell culture is inoculated with a seeding density of 10⁶ cells/mL in 1.5 L media. In exemplary aspects, the method comprises maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In exemplary aspects, the method comprises maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 30%, about 40%, about 50%, or about 60% oxygen saturation during the initial cell culture period.

The cell culture may be maintained in any culture medium. In exemplary aspects, the cell culture may be maintained in a medium suitable for cell growth and/or may be provided with one or more feeding media according to any suitable feeding schedule. In exemplary aspects, the method comprises maintaining the cell culture in a medium comprising glucose, lactate, ammonia, glutamine, and/or glutamate. In exemplary aspects, the method comprises maintaining the cell culture in a medium comprising manganese at a concentration less than about 1 μM during the initial cell culture period. In exemplary aspects, the method comprises maintaining the cell culture in a medium comprising about 0.25 μM to about 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb.

In exemplary embodiments, the type of cell culture is a fed-batch culture or a continuous perfusion culture. However, the methods of the invention are advantageously not limited to any particular type of cell culture.

### Recombinant Protein

In exemplary embodiments, the recombinant protein comprises an amino acid sequence comprising one or more N-glycosylation consensus sequences of the formula:

Asn-Xaa₁-Xaa₂

wherein Xaa₁ is any amino acid except Pro, and Xaa₂ is Ser or Thr.

In exemplary embodiments, the recombinant protein comprises a fragment crystallizable (Fc) polypeptide. The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. In exemplary embodiments, the recombinant protein comprises the Fc of an IgG, e.g., a human IgG. In exemplary aspects, the recombinant protein comprises the Fc an IgG1 or IgG2. In exemplary aspects, the recombinant protein is an antibody, a peptibody, or a Fc-fusion protein.

In exemplary aspects, the recombinant glycosylated protein is an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the invention include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody may be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody may be considered as a mammalian antibody, e.g., a mouse antibody, rabbit antibody, goat antibody, horse antibody, chicken antibody, hamster antibody, human antibody, and the like. In certain aspects, the recombinant protein is a human antibody. In certain aspects, the recombinant protein is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects, the recombinant glycosylated protein is an antibody fragment, e.g., a Fab, Fc, F(ab′)₂, or a pFc′, that retains at least one glycosylation site.

The architecture of antibodies has been exploited to create a growing range of alternative antibody formats that spans a molecular-weight range of at least 12-150 kDa and a valency (n) range from monomeric (n=1), dimeric (n=2) and trimeric (n=3) to tetrameric (n=4) and potentially higher; such alternative antibody formats are referred to herein as “antibody protein products”.

Antibody protein products include those based on antibody fragments, e.g., scFvs, Fabs and VHH/VH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the recombinant protein comprises any one of these antibody protein products. In exemplary aspects, the recombinant glycosylated protein is any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate.

The recombinant protein may be an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

The antibody protein product may lack certain portions of an antibody. However, generally, the fragment will comprise at least a portion of the Fc region of an antibody which is glycosylated post-translationally in eukaryotic cells.

Advantageously, the methods are not limited to the antigen-specificity of the antibody. Accordingly, the antibody has any binding specificity for virtually any antigen. In exemplary aspects, the antibody binds to a hormone, growth factor, cytokine, a cell-surface receptor, or any ligand thereof. In exemplary aspects, the antibody binds to a protein expressed on the cell surface of an immune cell. In exemplary aspects, the antibody binds to a cluster of differentiation molecule selected from the group consisting of: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79α, CD79β, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the antibody is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor I), U.S. Pat. Nos. 7,566,772 and 7,964,193 (interleukin-10), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publication No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In exemplary embodiments, the antibody is one of Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), Ibritumomab tiuxetan (product marketed with the brand name Zevalin®), I'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1.beta. antibodies such as canakinumab; anti-IL12/23 (p40) antibodies such as ustekinumab and briakinumab; and anti-IL2R antibodies, such as daclizumab. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies such as siltuximab; and anti-VEGF antibodies such as bevacizumab; anti-IL6 receptor antibodies such as tocilizumab.

### Compositions

Provided herein are compositions comprising increased amounts of afucosylated glycoforms of a protein. In exemplary embodiments, the compositions are prepared by the inventive methods of preparing a composition comprising afucosylated glycoforms of a protein produced reaction with fucosidase, described herein. In exemplary aspects, at least about 10% of the protein in the composition is an afucosylated glycoform. In exemplary aspects, at least about 20% of the protein in the composition is an afucosylated glycoform. In exemplary aspects, at least about 30% of the protein in the composition is an afucosylated glycoform. In exemplary aspects, at least about 40% of the protein in the composition is an afucosylated glycoform. In exemplary aspects, at least about 50% of the protein in the composition is an afucosylated glycoform. In exemplary aspects, at least about 60% of the protein in the composition is an afucosylated glycoform. In exemplary aspects, at least about 70% of the protein in the composition is an afucosylated glycoform. In exemplary aspects, at least about 80% of the protein in the composition is an afucosylated glycoform. In exemplary aspects, at least about 90% of the protein in the composition is an afucosylated glycoform. In exemplary aspects, greater than about 90% or greater than about 95% of the protein in the composition is an afucosylated glycoform.

In exemplary aspects, the methods of the disclosure increase the percentage of aglycosylated glycoforms by 2% or more. In exemplary aspects, the methods of the invention increase the percentage of afucosylated glycoforms by 5% or more. In exemplary aspects, the methods of the invention increase the percentage of afucosylated glycoforms by 10% or more.

The compositions of the invention are, in exemplary aspects, pharmaceutical composition. In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the US Pharmacopeia for use in animals, including humans.

The pharmaceutical composition can comprise any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety; and Remington's Pharmaceutical Sciences, Sixteenth Edition, E. W. Martin (Mack Publishing Co., Easton, Pa., 1980), which is incorporated by reference in its entirety

In exemplary aspects, the pharmaceutical composition comprises formulation materials that are nontoxic to recipients at the dosages and concentrations employed. In specific embodiments, pharmaceutical compositions comprising a therapeutically effective amount of afucosylated glycoforms of a protein and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18^{th} Edition, (A. R. Genrmo, ed.), 1990, Mack Publishing Company.

The pharmaceutical compositions may be formulated to achieve a physiologically compatible pH. In some embodiments, the pH of the pharmaceutical composition may be for example between about 4 or about 5 and about 8.0 or about 4.5 and about 7.5 or about 5.0 to about 7.5. In exemplary embodiments, the pH of the pharmaceutical composition is between 5.5 and 7.5.

The following examples are given merely to illustrate the present invention and not in any way to limit its scope.

### EXAMPLES

### Example 1

The ability of various fucosidases to impact levels of afucosylation on an IgG1 monoclonal antibody was studied. One laboratory has reported the successful removal of the core α1,6- and α1,3-linked core fucose on N-linked glycoproteins with a broad-specificity fucosidase O. See Vainauskas et al., 2018, Nature, 8:9504. Unless otherwise indicated, all chemicals, reagents and solvents were sourced from Sigma-Aldrich (St. Louis, MO).

Three different fucosidases were examined in an effort to identify a novel α1-6-fucosidase with the ability to remove core fucose from unlabeled N-glycans attached to mAbs:

Hfuc: α-(1-2,3,4,6)-L-Fucosidase (Homo sapiens), synthesized using a recombinant microbial expression system, is a broad specificity fucosidase with an optimum pH at pH 4.0 and a temperature optimum of 50° C. (Megazyme, Bray, Ireland; E-FUCHS). See Liu et al., 2009, Biochemistry 48:110-120.

BKF: α1-2,3,4,6 Fucosidase from Bovine Kidney (BKF) expressed in E. coli is a broad specificity exoglycosidase that cleaves α1-2 and α1-6 fucose residues more efficiently than other linkages and has slight activity towards α1-3 fucose residues (New England Biolabs, Ipswich, MA; Cat. No. P0748S). See Vainauskas et al., 2018, Nature, 8:9504.

FucO: α1-2,4,6 Fucosidase O is a broad specificity exoglycosidase cloned from Omnitrophica bacterium and expressed in E. coli that catalyzes the hydrolysis of fucose connected with a α1-2, α1-4 and α1-6 from oligosaccharides. It favorably cleaves α1-6 fucose residues than other linkages. The optimum reaction temperature for this enzyme is 50° C. and it is highly active from pH 4.0-6.0 with optimal activity at pH 5.5. (New England Biolabs, Ipswich, MA; Cat. No. P07449S). See Vainauskas et al., 2018, Nature, 8:9504.

A human anti-IL-12 IgG1 mAb was sourced from a 2000L large scale facility at FujiFilm Diosynth Bioprocesses, North Carolina (FDBU). The harvested cell culture from FDBU was processed through an affinity chromatography column to clear out the residual host cell proteins, DNA and cell debris. The purified mAb pool was virus inactivated (VI) at a low pH and processed through depth filtration at FDBU prior to receipt. The protein pool was stored at −70° C. The protein pool and enzyme stocks were thawed in a Polyscience AD285150-A11B heated circulator water bath. The proteins were sterile filtered using 0.22 μm pore size filter (Stericup®, Millipore Sigma) prior to use.

The in vitro experiments on intact unlabeled mAbs human anti-IL12 IgG1 antibody were conducted at the pH optimum for each fucosidase as per the manufacturer's specifications and at a temperature of 36° C. FucO and BKF were tested at an enzyme level of 100,000 U/mmol mAb, and Hfuc was tested at 10,000 U/mmol mAb based on the manufacturers' definitions of enzyme unit. A control condition was tested without the presence of any enzyme. The intact mAb present in the VI pool comprises an α-1,6-fucosylated core glycan structure (substrate for the glycoenzymes) at a concentration in the range of 18 to 20 mg/mL of mAb protein. Table 1 below shows a summary of the design of the experiment.

TABLE 1
Experimental Design

| | Fucosidase | | Fucosidase Level |
|---|---|---|---|
| Condition | Enzyme | pH | (U/mmol mAb) |
| 1 | N/A | 5.0 | 0 (control) |
| 2 | Hfuc | 4.0 | 10,000 |
| 3 | BKF | 5.0 | 100,000 |
| 4 | FucO | 5.0 | 100,000 |

To begin the study, approximately 10 mL of the mAb1 VI pool was warmed to room temperature. The pool was then aliquoted into one pool of 4 mL and three pools of 2 mL. A 2 mL sample was taken from condition 1 immediately and frozen at −70° C. Appropriate amounts of fucosidase according to Table 1 were added to each of the remaining conditions. Fucosidase was diluted according to Table 1 with 100 mM sodium acetate, pH 5.0, to ensure addition of accurate amounts of fucosidase. All conditions were moved to the 36° C. incubator after enzyme addition.

2 mL samples from each condition were taken at the 24 hr time point. Each sample was frozen at −70° C. until submission for HILIC analysis. One 1 mL sample from each condition was submitted for HILIC analysis once the study execution had been completed.

The glycan map of enzymatically released N-linked glycans was determined using HILIC. N-linked glycans on mAbs were released enzymatically using PNGase F protein in a sodium phosphate buffer (pH 7.5) for ˜2 hours at ˜37° C. on a BEH Glycan Column, 2.1×150 mm, 1.7 μm (Waters, Catalog #186004742). The glycans are labeled with 2-aminobenzoic acid (2-AA) and sodium cyanoborohydride, incubated at −80° C. for about 75 minutes and separated by HILIC (hydrophilic interaction liquid chromatography) with an in-line fluorescence detector. The total glycan % of Afucosylated, High Mannose, Sialylated and β-Galactosylated species were calculated by integrating the individual glycan peaks.

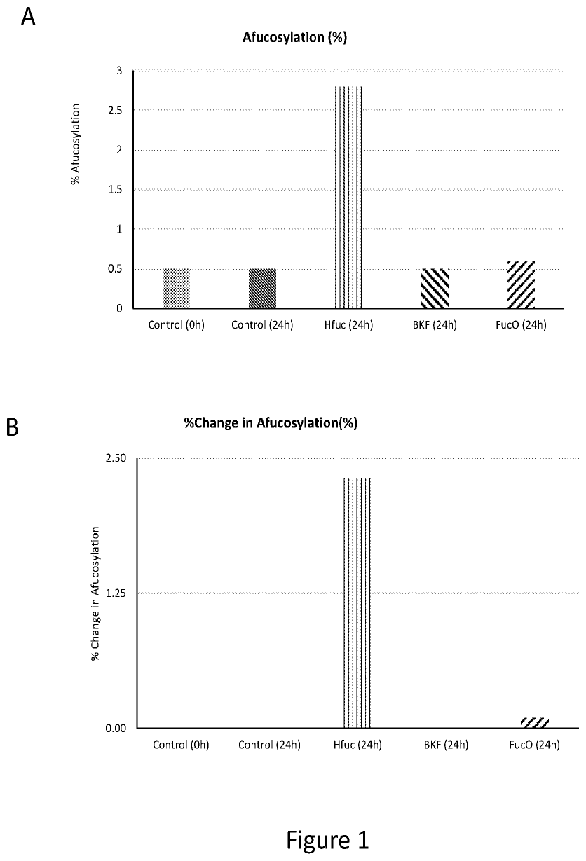

The results for afucosylation are shown in FIG. 1A. The control sample showed no variations in the level of afucosylation (%) after 24 hr as expected due to absence of fucosidase. In contrast, the Hfuc sample shows a steep increase in afucosylation levels indicating that the enzyme was successful in cleaving off the α1-6-linked fucose on mAb. Surprisingly, the levels of afucosylation did not show any significant change for the BKF and FucO samples. The small change observed for FucO could be associated with the variability of the glycan analysis assay. FIG. 1B displays the % change in afucosylation (%) more distinctly when plotted against the control and fucosidase tested. The Hfuc sample displayed a 2.3% increase in the level of afucosylation (%) in comparison to the control.

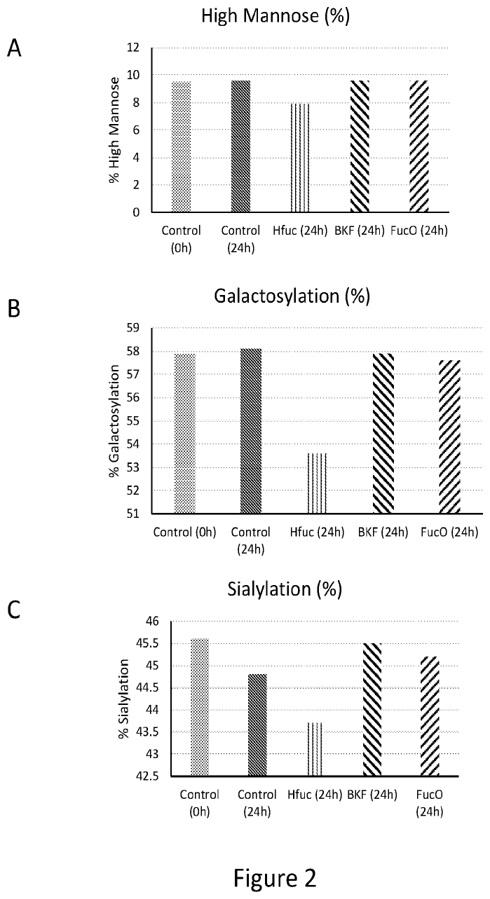

The results for high mannose, galactosylation, and sialylation are shown in FIGS. 2A, 2B and 2C, respectively. After 24 hr incubation, BKF and FucO had no effect on high mannose (HM) (similar to controls), while Hfuc decreased high mannose to 7.9% instead of 9.5%. This may be because of the absence of a co-factor such as Zn or Ca which was in buffers used with BKF and FucO but not in the buffer used with Hfuc. Hfuc also decreased percent galactosylation by ˜4.5% and percent sialylation by ˜1%, where BKF and FucO had minimal effects on galactosylation and sialylation. This suggests that BKF and FucO did not react with fucosylated mAb.

This study demonstrates that Hfuc fucosidase from Homo sapiens exhibits a potential to manipulate the core α1-6-linked fucose on N-glycans attached to mAbs. A 2.3% increase in afucosylation levels for the sample with Hfuc was seen after 24 hours incubation at pH 4.0 and a temperature of 36° C. The specificity and efficacy of this enzyme towards α1-6 fucosylated moieties could be associated with its usual function within the complex mammalian body environment.

This suggests an alternative enzymatic solution for modifying the glycan levels on mAbs without the complexities involved during upstream development.

All references, including publications, patent applications, and patents, cited herein are hereby incorporated by reference to the same extent as if each reference were individually and specifically indicated to be incorporated by reference and were set forth in its entirety herein.

1. A method for obtaining a recombinant glycosylated protein having increased levels of afucosylated glycoforms, the method comprising
1) incubating a purified recombinant glycosylated protein with a human broad specificity fucosidase in a buffer suitable for fucosidase activity for a time and under conditions suitable to increase afucosylation of the recombinant glycosylated protein; and
2) separating the recombinant glycosylated protein having increased levels of afucosylated glycoforms from the fucosidase;
wherein the recombinant glycosylated protein is not reacted with a glycosyltransferase or sialyltransferase.

2. The method of claim 1, wherein the human fucosidase is α-(1-2,3,4,6)-L-fucosidase.

3. The method of claim 1, wherein the fucosidase is present at a level between 1000 U/mmol to 100,000 U/mmol recombinant glycosylated protein.

4. The method of claim 3, wherein the fucosidase is present at a level between 5,000 U/mmol to 25,000 U/mmol recombinant glycosylated protein.

5. The method of claim 1, wherein said incubating is for between 1 hour to 24 hours.

6. The method of claim 1, wherein the buffer has a pH from about 4.0 to about 5.0.

7. The method of claim 1, wherein the temperature is selected from a temperature between 30° C. and 40° C.

8. The method of claim 7, wherein the temperature is selected from a temperature between 35° C. and 38° C.

9. The method of claim 1, wherein the buffer is sodium acetate, phosphate buffered saline or MES.

10. The method of claim 1, wherein the purified recombinant glycosylated protein is in an amount at least 10 g/L

11. The method of claim 1, wherein said fucosidase is immobilized on a solid phase.

12. The method of claim 11, wherein the solid phase is a protein A chromatography resin.

13. The method of claim 1, wherein the purified recombinant glycosylated protein has been purified by one or more chromatography steps.

14. The method of claim 1, wherein the levels of one or more of A1G0, A2G0, A2G1a, A2G1b, A2G2, and A1G1M5 of the recombinant glycosylated protein are increased.

15. The method of claim 1, wherein the levels of high mannose (HM) glycoforms of the recombinant glycosylated protein are decreased.

16. The method of claim 15, wherein the levels of one or more of Man5, Man6, Man7, Man8, and/or Man9 of the recombinant glycosylated protein are decreased.

17. The method of claim 1, wherein the percent galactosylation is reduced.

18. The method of claim 1, wherein the percent sialylation is reduced.

19. The method of claim 1, wherein the recombinant glycosylated protein is separated from the fucosidase using one or more purification steps.

20. The method of claim 19, wherein the one or more purification steps are selected from diafiltration, ultrafiltration, and sterile filtration.

21. The method of claim 1, wherein the recombinant glycosylated protein is an antibody, a peptibody, or a Fc-fusion protein.

22. The method of claim 21, wherein the recombinant glycosylated protein is an antibody that binds to CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79α, CD79β, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, CD182, erythropoietin, IL-2, IL-3, IL-4, IL-5, IL-6, IL-7, IL-9, IL-11, IL-13, G-CSF, IL-15, GM-CSF, OSM, IFNgamma, IFNα, IFNβ, TNFα, TNFβ, LTβ, CD40 ligand, Fas ligand, CD27 ligand, CD30 ligand, 4-BBL, TGFβ, IL-1α, IL-1β, IL-1 RA, IL-10, IL-12, MIF, IL-16, IL-17, IL-18, glucagon receptor, IL-17 receptor A, Sclerostin, IGF-1 receptor, myostatin, epidermal growth factor receptor, SARS coronavirus, OPGL, Angiopoietin-2, NGF, TGF-β type II receptor, connective tissue growth factor, properdin, CTLA-4, interferon-gamma, MAdCAM, amyloid, insulin-like growth factor I, interleukin-1β, c-Met, M-CSF, MUC18, interleukin-4 receptor, fibroblast growth factor-like polypeptides, α-4 β-7, Activin Receptor-like Kinase-1, Activin A, angiopoietin-1, angiopoietin-2, C-FMS, galanin, insulin like growth factor, LDCAM, DKK1, osteoprotegerin, OV064, PSMA, PAR2, HEPCIDIN, B7L-1, c-Kit, ULBP, TSLP, SIGIRR, HER-3, ataxin-1-like polypeptide, TNF-α converting enzyme, IL1-R1, TGF-β type II receptor, TNF receptor-like molecules, connective tissue growth factor, TRAIL receptor-2, erythropoietin receptor, B7RP1, properdin, RANKL, carbonic anhydrase IX (CA IX) tumor antigen, parathyroid hormone, ACPL, monocyte chemo-attractant protein-1, SCF, 4-1BB, PDGFD, Flt-3 ligand, metalloproteinase inhibitor, LERK-5, LERK-6, brain-derived neurotrophic factor, epithelium-derived T-cell factor, neurotrophic factor NNT-1, proprotein convertase subtilisin kexin type 9 (PCSK9), IL-18 RECEPTOR, or C-FMS.

23. The method of claim 21, wherein the recombinant protein is one of Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-1131 (product marketed with the brand name Bexxar®), Efalizumab (product marketed with the brand name Raptiva®), Cetuximab (product marketed with the brand name Erbitux®), Ibritumomab tiuxetan (product marketed with the brand name Zevalin®), Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), Pertuzumab (product marketed with the brand name Perjeta®), adalimumab, infliximab, etanercept, golimumab, certolizumab pegol; canakinumab; ustekinumab, briakinumab; daclizumab, belimumab; epratuzumab; daclizumab; iratumumab, gemtuzumab, alemtuzumab; ipilimumab; cetuximab; trastuzumab, pertuzumab; siltuximab; bevacizumab; and tocilizumab.
